(19) United States
(12) Patent Application Publication
(10) Pub. No.: US 2025/0382367 A1
(43) Pub. Date: Dec. 18, 2025

(54) NANOBODY TARGETING CD150 PROTEIN AND USE THEREOF

(71) Applicant: UNIVERSITY OF SCIENCE AND TECHNOLOGY OF CHINA, Hefei (CN)

(72) Inventors: Cheng SUN, Hefei (CN); Guojing YAO, Hefei (CN)

(21) Appl. No.: 19/315,651

(22) Filed: Aug. 31, 2025

Related U.S. Application Data
(63) Continuation of application No. PCT/CN2024/073555, filed on Jan. 23, 2024.

(30) Foreign Application Priority Data
Mar. 15, 2023 (CN) 202310268767.1

(51) Int. Cl. C07K 16/28; A61K 39/00; A61P 35/00

(52) U.S. Cl. CPC C07K 16/2803; A61P 35/00; A61K 2039/505; C07K 2317/24; C07K 2317/52; C07K 2317/569; C07K 2317/92

(57) ABSTRACT

Provided is a nanobody or antigen-binding fragment capable of specifically recognizing CD150. The antibody or antigen-binding fragment comprises heavy chain variable region CDR sequences selected from at least one of following sequences: SEQ ID NO: 1 to SEQ ID NO: 3, or amino acid sequences having at least 80% identity with same. The antibody has low immunogenicity, and can specifically target and bind CD150. By means of blocking the function of CD150 and a ligand thereof, the antibody can reverse the function depletion of immune cells, thereby achieving the effect of killing tumor cells.

### CROSS-REFERENCE TO RELATED APPLICATIONS

This application is a continuation of International Patent Application No. PCT/CN2024/073555 filed on Jan. 23, 2024, which claims priority to and benefits of patent application No. 202310268767.1, filed with China National Intellectual Property Administration on Mar. 15, 2023, the entire contents of which are incorporated herein by reference.

### STATEMENT REGARDING SEQUENCE LISTING

A Sequence Listing associated with this application is being filed concurrently herewith in ASCII format and is hereby incorporated by reference into the present specification. The text file containing the Sequence listing is titled “Sequence_Listing.xml”, was created on Aug. 8, 2025, and is 10,982 bytes in size.

### FIELD

The present disclosure relates to the field of biotechnology. Particularly, the present disclosure relates to a nanobody targeting CD150 protein and use thereof, and more particularly, to an antibody or antigen-binding fragment, a recombinant protein, a nucleic acid molecule, an expression vector, a recombinant cell, a pharmaceutical composition, a pharmaceutical use, a kit for detecting CD150, and a method for treating a disease and use thereof.

### BACKGROUND

Malignant tumors are diseases that seriously threaten human life and health. Currently available treatment strategies for malignant tumors, including surgical resection, radiotherapy, chemotherapy, small-molecule targeted therapy, immune checkpoint therapy, and cell and gene therapy, only exert limited efficacy in certain patients with malignant tumors. Thus, malignant tumors remain a major challenge regarding human life and health. In the treatment of tumor patients, surgical resection is generally suitable only for early-stage patients and carries the risk of poor recovery and complications. Radiotherapy and chemotherapy used in more advanced stages may offer therapeutic benefits but are also associated with disadvantages such as toxicity and drug resistance. Among immunotherapies, immune checkpoint therapy is commonly employed to inhibit immune checkpoint pathways and reverse tumor immune evasion mechanisms, thereby enabling tumor cell killing. For the above reasons, it is necessary to develop immune checkpoint antibodies against specific targets.

Conventional monoclonal antibodies are composed of two glycosylated heavy chains and two non-glycosylated light chains. These molecules have large molecular weight, complex production processes, and limited amenability to engineering and modification. In contrast, camelids naturally produce a class of antibodies composed only of two heavy chains, known as heavy-chain antibodies. The variable domain of these antibodies is composed of two identical heavy chain variable regions, and such antibodies are referred to as single-domain antibodies (sdAbs). Single-domain antibodies typically have diameters of less than 10 nanometers and are therefore also called nanobodies. In alpacas, these single-domain antibodies are referred to as VHH. Compared to conventional scFv or Fab fragments, single-domain antibodies offer numerous advantages, including smaller molecular weight, higher penetration, improved stability and solubility, and glycosylation-independent functionality. Additionally, single-domain antibodies usually possess an extended complementarity-determining region 3 (CDR3), which can form a convex structural surface to recognize antigenic epitopes. This allows them to recognize hidden antigenic epitopes that are difficult for traditional antibodies to access.

CD150 is a type I single-pass transmembrane phosphoglycoprotein with a molecular weight ranging from 70 kDa to 95 kDa. The core protein of CD150 has a molecular weight of approximately 42 kDa. There are two immunoreceptor tyrosine-based switch motifs (ITSMs) in its cytoplasmic tail. CD150 is expressed on the surface of malignant cells and serves as a target for measles virus (MV)-mediated oncolysis and potentially as a target for antibody-based therapy. During tumor progression, CD150 is highly expressed on B cells, and its interaction with CD8⁺ T cells leads to T cell exhaustion, thereby limiting the killing effect on the tumors.

Therefore, there is an urgent need in the art to develop a nanobody targeting CD150 to overcome the drawbacks of conventional tumor therapies, such as severe side effects and secondary drug resistance.

### SUMMARY

The present disclosure aims to solve one of the technical problems in the related art at least to some extent. To this end, the present disclosure provides an anti-CD150 nanobody, which exhibits high binding activity to CD150. The nanobody reverses the functional exhaustion of T cells, B cells, and NK cells by blocking the interaction between CD150 and its ligand, thereby achieving an effect of killing tumor cells.

In a first aspect of the present disclosure, the present disclosure provides an antibody or antigen-binding fragment. According to embodiments of the present disclosure, the antibody includes a heavy chain variable region CDR sequence selected from at least one of SEQ ID NO: 1 to SEQ ID NO: 3 or amino acid sequences having at least 80% identity to SEQ ID NO: 1 to SEQ ID NO: 3.

According to the embodiments of the present disclosure, the antibody or antigen-binding fragment can bind to CD150 and thus block the interaction between CD150 and its ligand, thereby reversing the functional exhaustion of T cells and further achieving the effect of killing tumor cells. In addition, the antibody or antigen-binding fragment thereof exhibits high specificity and can thus avoid the occurrence of secondary drug resistance.

(SEQ ID NO: 1)
GRAFSDYAVG.
(SEQ ID NO: 2)
EFVATISWSGFSTY.
(SEQ ID NO: 3)
ADRNGTSFPYIRRDEYDY

According to the embodiments of the present disclosure, the above-mentioned antibody or antigen-binding fragment may further include at least one of the following additional technical features.

According to an embodiment of the present disclosure, the antibody or antigen-binding fragment includes a heavy chain variable region CDR1 sequence, a heavy chain variable region CDR2 sequence, and a heavy chain variable region CDR3 sequence as set forth in amino acid sequences of SEQ ID NO: 1, SEQ ID NO: 2, and SEQ ID NO: 3, respectively, or amino acid sequences having at least 80% identity to SEQ ID NO: 1, SEQ ID NO: 2, and SEQ ID NO: 3, respectively.

According to an embodiment of the present disclosure, the antibody or antigen-binding fragment further includes a heavy chain framework region sequence. At least a portion of the heavy chain framework region sequence is from at least one of a murine antibody, a human antibody, a primate antibody, an alpaca antibody, or a mutant thereof.

It should be noted that the “antibody or antigen-binding fragment thereof” described in the present disclosure may also be humanized.

As used herein, the term “humanized” or “humanization” refers to modifications of the amino acids of antibodies or antigen-binding fragments that can reduce immunogenicity, including amino acid mutations, insertions, deletions, conjugation of chemical groups, and the like.

Exemplarily, the humanization is typically accomplished by framework grafting and protein surface amino acid humanization.

The above-mentioned humanized antibody according to the embodiments of the present disclosure exhibits reduced immunogenicity, while retaining the ability to specifically bind to CD150 and block the binding between CD150 and anti-CD150 antibodies.

According to an embodiment of the present disclosure, the antibody or antigen-binding fragment includes a heavy chain variable region having an amino acid sequence as set forth in SEQ ID NO: 4.

(SEQ ID NO: 4)
QVQLVESGGGLVQAGGSLRLSCAASGRAFSDYAVGWFRQAPGREREFV
ATISWSGFSTYYADSVKGRFTISRDNAEKTVTLQMNSLKPEDTAVYYC
AADRNGTSFPYIRRDEYDYWGQGTQVT.

According to an embodiment of the present disclosure, the antibody or antigen-binding fragment further includes a heavy chain constant region. At least a portion of the heavy chain constant region is from at least one of a murine antibody, a human antibody, a primate antibody, or a mutant thereof.

According to an embodiment of the present disclosure, the heavy chain constant region is from an alpaca antibody. The inventors found that the use of alpaca single-domain antibodies offers broader applicability and reduced immunogenicity.

According to an embodiment of the present disclosure, the antibody or antigen-binding fragment includes a monoclonal antibody or a polyclonal antibody.

According to an embodiment of the present disclosure, the monoclonal antibody includes at least one of a Fab antibody, an Fv antibody, a single-chain antibody, a single-domain antibody, and a minimum recognition unit.

According to an embodiment of the present disclosure, the antibody has an amino acid sequence as set forth in SEQ ID NO: 5.

(SEQ ID NO: 5)
QVQLVESGGGLVQAGGSLRLSCAASGRAFSDYAVGWFRQAPGREREFVA
TISWSGFSTYYADSVKGRFTISRDNAEKTVTLQMNSLKPEDTAVYYCAA
DRNGTSFPYIRRDEYDYWGQGTQVT.

In a second aspect of the present disclosure, the present disclosure provides a recombinant protein. According to embodiments of the present disclosure, the recombinant protein includes the antibody or antigen-binding fragment according to the first aspect of the present disclosure. The recombinant protein according to the embodiments of the present disclosure is capable of specifically binding to CD150 with high CD150 binding activity, and can be used to detect CD150, as well as to block the binding of CD150 to its antibody, thereby blocking signal transduction and further inhibiting tumor proliferation.

According to an embodiment of the present disclosure, the above recombinant protein may further include at least one of the following additional technical features.

According to an embodiment of the present disclosure, the recombinant protein further includes at least one selected from a bioactive protein or fragment thereof, and a bioactive polypeptide or fragment thereof.

According to an embodiment of the present disclosure, the bioactive protein or fragment thereof includes at least one selected from a protein tag, a protein toxin or fragment thereof, a tumor necrosis factor or fragment thereof, an interferon or fragment thereof, a biological response modifier or fragment thereof, and an Fc fragment.

As used herein, the term “protein tag” generally refers to a polypeptide or protein that is expressed in fusion with a target protein (such as an antibody or antigen-binding fragment), and can be used for an expression, detection, tracking, or purification of the target protein. Examples include, but are not limited to, a His tag (with the sequence HHHHHH), a Flag tag (with the sequence DYKDDDDK), a GST tag (also known as glutathione S-transferase tag), an MBP tag (also known as maltose-binding protein tag), a SUMO tag, and a C-Myc tag.

As used herein, the term “toxin” generally refers to substances toxic to the host, including protein toxins and non-protein toxins. The protein toxins include but are not limited to abrin, ricin A, Pseudomonas exotoxin, and diphtheria toxin. In the present disclosure, the protein toxin is preferably a protein toxin having enzymatic activity.

As used herein, the “tumor necrosis factor” generally refers to substance capable of inducing hemorrhagic necrosis in various tumors, including but not limited to TNF-α and TNF-β.

As used herein, the term “interferon” generally refers to a class of glycoproteins capable of directly killing or inhibiting viruses. Examples include, but are not limited to, IFN-α, IFN-β, and IFN-γ.

As used herein, the term “biological response modifier” generally refer to a class of protein substances that directly or indirectly enhance the body's anti-tumor effects via the immune system. Examples include, but are not limited to, lymphokines, IL-2, IL-6, IL-10, and GM-CSF.

As used herein, the term “Fc fragment” generally refers to the Fc region from IgG (e.g., IgG1, IgG2, IgG3, or IgG4 subtype), IgA1, IgA2, IgD, IgE or IgM, including CH2 and CH3 region, and optionally a hinge region. Preferably, IgG, IgA1, IgA2, IgD, IgE, or IgM is from a murine, human, primate, or alpaca source.

In a third aspect of the present disclosure, the present disclosure provides a nucleic acid molecule. According to embodiments of the present disclosure, the nucleic acid molecule encodes the antibody or antigen-binding fragment according to the first aspect of the present disclosure or the recombinant protein according to the second aspect of the present disclosure. The nucleic acid molecules according to the embodiments of the present disclosure encode the aforementioned antibodies or antigen-binding fragments, and recombinant proteins.

According to an embodiment of the present disclosure, the nucleic acid molecule is DNA.

It should be noted that those skilled in the art should understand that nucleic acids mentioned in the present disclosure actually include any one or two of complementary double strands. For convenience, in the present description and the claims, although only one strand is given in most cases, the other strand complementary to the one strand is also disclosed actually. In addition, nucleotide sequences in the present disclosure include a DNA form or an RNA form. When one form is disclosed, it means that the other form is also disclosed.

In a fourth aspect of the present disclosure, the present disclosure provides an expression vector. According to embodiments of the present disclosure, the expression vector carries the nucleic acid molecule according to the third aspect of the present disclosure. When the above-mentioned nucleic acid molecule is linked to a vector, the nucleic acid molecule may be directly or indirectly linked to control components on the vector, as long as these control components can control translation and expression of the nucleic acid molecule. Of course, these control components may be directly from the vector itself, or they may be exogenous, that is, not from the vector itself. Of course, the nucleic acid molecule may be operably linked to the control components. As used herein, the term “operably linked” means that an exogenous gene is linked to a vector, enabling control components in the vector, such as a transcriptional control sequence and a translational control sequence, to exert an expected effect of regulating transcription and translation of the exogenous gene. Commonly vectors, for instance, may be plasmids, phages, etc. After the expression vector according to some specific embodiments of the present disclosure is introduced into a suitable recipient cell, the aforementioned antibody or antigen-binding fragment or recombinant protein can be effectively expressed under mediation of a regulatory system, thereby realizing large-scale in vitro production of the antibody or antigen-binding fragment or recombinant protein.

According to an embodiment of the present disclosure, the expression vector is a eukaryotic expression vector or a prokaryotic expression vector.

According to an embodiment of the present disclosure, the expression vector is a plasmid expression vector.

In a fifth aspect of the present disclosure, the present disclosure provides a recombinant cell. According to embodiments of the present disclosure, the recombinant cell carries the nucleic acid molecule according to the third aspect of the present disclosure or the expression vector according to the fourth aspect of the present disclosure, or expresses the antibody or antigen-binding fragment according to the first aspect of the present disclosure or the recombinant protein according to the second aspect of the present disclosure. The recombinant cell can be used to efficiently express the aforementioned antibodies or antigen-binding fragments, or recombinant proteins in the cells under suitable conditions.

It should be noted that the “suitable conditions” described in the specification of the present disclosure refer to conditions suitable for the expression of the antibody or antigen-binding fragment, or the recombinant protein according to the present disclosure. Those skilled in the art can easily conceive that, the conditions suitable for the expression of the antibody or antigen-binding fragment or the recombinant protein include, but not limited to, a suitable transformation or transfection mode, a suitable transformation or transfection condition, a healthy host cell state, a suitable density of host cells, a suitable culture environment, and suitable culture period. The “suitable conditions” are not specially limited. The most suitable conditions for the expression of the antibody or antigen-binding fragment or the recombinant protein may be optimized by those skilled in the art based on specific laboratory environment.

According to an embodiment of the present disclosure, the recombinant cell is obtained by introducing the expression vector according to the fourth aspect of the present disclosure into a host cell.

According to an embodiment of the present disclosure, the recombinant cell is a eukaryotic cell.

According to an embodiment of the present disclosure, the recombinant cell is a mammalian cell.

In a sixth aspect of the present disclosure, the present disclosure provides a pharmaceutical composition. According to embodiments of the present disclosure, the pharmaceutical composition includes the antibody or antigen-binding fragment according to the first aspect of the present disclosure, the recombinant protein according to the second aspect of the present disclosure, the nucleic acid molecule according to the third aspect of the present disclosure, the expression vector according to the fourth aspect of the present disclosure, or the recombinant cell according to the fifth aspect of the present disclosure. The pharmaceutical composition according to the present disclosure is capable of binding to CD150 and thus block binding of CD150 to its receptor, which can effectively treat or prevent tumor proliferation.

According to an embodiment of the present disclosure, the pharmaceutical composition further includes a pharmaceutically acceptable carrier or excipient.

It should be noted that the “pharmaceutically acceptable carrier” may include any and all solvents that are physiologically compatible. Specific examples may be one or more of water, saline, phosphate buffered saline, and glucose, and combinations thereof. In many cases, the pharmaceutical composition includes isotonic agents, such as sodium chloride. Of course, the “pharmaceutically acceptable carrier” may also include trace amounts of auxiliary substances, such as buffers, to prolong shelf life or efficacy of the antibody.

For example, the antibody or antigen-binding fragment according to the present disclosure may be incorporated into pharmaceutical compositions suitable for parenteral administration (e.g., intravenous, subcutaneous, intraperitoneal, intramuscular). These pharmaceutical compositions may be prepared in various forms, such as liquid, semi-solid, and solid dosage forms, including but not limited to liquid solutions (e.g., injection solutions and infusion solutions) and liposomes. Typical pharmaceutical compositions are in the form of injectable or infusible solutions. The antibody may be administered by intravenous infusion or injection, or by intramuscular or subcutaneous injection.

In a seventh aspect of the present disclosure, the present disclosure provides use of the antibody or antigen-binding fragment according to the first aspect of the present disclosure, the recombinant protein according to the second aspect of the present disclosure, the nucleic acid molecule according to the third aspect of the present disclosure, the expression vector according to the fourth aspect of the present disclosure, the recombinant cell according to the fifth aspect of the present disclosure, or the pharmaceutical composition according to the sixth aspect of the present disclosure in the manufacture of a medicament for treating or preventing a tumor.

According to an embodiment of the present disclosure, the tumor includes lung cancer, gastric cancer, colorectal cancer, liver cancer, breast cancer, esophageal cancer, skin cancer, prostate cancer, renal cancer, endometrial cancer, cervical cancer, thyroid cancer, and hematological tumors.

In an eighth aspect of the present disclosure, the present disclosure provides a kit. According to embodiments of the present disclosure, the kit includes the antibody or antigen-binding fragment according to the first aspect of the present disclosure, the recombinant protein according to the second aspect of the present disclosure, the nucleic acid molecule according to the third aspect of the present disclosure, the expression vector according to the fourth aspect of the present disclosure or the recombinant cell according to the fifth aspect of the present disclosure. The antibody or antigen-binding fragment in the kit according to the present disclosure can effectively bind to CD150. In addition, under suitable conditions, the nucleic acid molecule, the expression vector, or the recombinant cell can express the antibody or antigen-binding fragment. Further, the kit including the above-mentioned substances can effectively bind to CD150 and can be used to effectively detect CD150. The kit can be used for scientific research, such as qualitatively or quantitatively detecting CD150 in biological samples, and can also be used to determine the state of an individual. For example, after obtaining a CD150 level of the individual, the kit can determine whether the CD150 level is excessively higher or excessively lower than a normal level. The biological samples may be cells, tissues, or blood.

In a ninth aspect of the present disclosure, the present disclosure provides use of the antibody or antigen-binding fragment according to the first aspect of the present disclosure, the recombinant protein according to the second aspect of the present disclosure, the nucleic acid molecule according to the third aspect of the present disclosure, the expression vector according to the fourth aspect of the present disclosure, or the recombinant cell according to the fifth aspect of the present disclosure in the preparation of a kit for detecting CD150.

According to the embodiments of the present disclosure, the kit according to the present disclosure may be a kit using specific binding properties of CD150 antigen and antibody for, for example, immunoblotting or immunoprecipitation. These kits may include any or several of antagonists, anti-CD150 antibodies, or medicament reference materials; protein purification columns; immunoglobulin affinity purification buffering agents; cell determination diluents; manual or literatures. The anti-CD150 antibodies may be used for diagnostic tests of different types, for example, for detecting various diseases, or the existence of medicaments, toxins or other proteins in vitro or in vivo; for instance, for diagnosing related diseases by detecting serum or blood from subjects.

In a tenth aspect of the present disclosure, the present disclosure provides a method for treating or preventing a disease. According to embodiments of the present disclosure, the method includes: administering to a subject the antibody or antigen-binding fragment according to the first aspect of the present disclosure, the recombinant protein according to the second aspect, the nucleic acid molecule according to the third aspect, the expression vector according to the fourth aspect, the recombinant cell according to the fifth aspect, or the pharmaceutical composition according to the sixth aspect. According to an embodiment of the present disclosure, the administration of the antibody or antigen-binding fragment, the recombinant protein nucleic acid molecule, the expression vector, the recombinant cell, or the pharmaceutical composition according to any one of the embodiments of the present disclosure can be used to treat or prevent a disease.

According to an embodiment of the present disclosure, the disease includes lung cancer, gastric cancer, colorectal cancer, liver cancer, breast cancer, esophageal cancer, or thyroid cancer.

In an eleventh aspect of the present disclosure, the present disclosure provides use of the antibody or antigen-binding fragment according to the first aspect, the recombinant protein according to the second aspect, the nucleic acid molecule according to the third aspect, the expression vector according to the fourth aspect, the recombinant cell according to the fifth aspect, or the pharmaceutical composition according to the sixth aspect in the treatment or prevention of a disease. According to an embodiment of the present disclosure, the antibody or antigen-binding fragment, the recombinant protein nucleic acid molecule, the expression vector, the recombinant cell, or the pharmaceutical composition used in any one of the embodiments of the present disclosure can be used to treat or prevent a disease.

According to an embodiment of the present disclosure, the disease includes lung cancer, gastric cancer, colorectal cancer, liver cancer, breast cancer, esophageal cancer, or thyroid cancer.

Additional aspects and advantages of the embodiments of the present disclosure will be provided at least in part in the following description, or will become apparent in part from the following description, or can be learned from the practice of the embodiments of the present disclosure.

### BRIEF DESCRIPTION OF THE DRAWINGS

These and other aspects and advantages of embodiments of the present disclosure will become apparent and more readily appreciated from the following descriptions made with reference to the accompanying drawings.

FIG. 1 shows the expression, purification, and characterization of the CD150 immunogen according to Example 1 of the present disclosure.

FIG. 2 shows the technical process for immunizing alpacas with the CD150 protein according to Example 1 of the present disclosure.

FIG. 3 shows the monoclonal phage ELISA (enzyme-linked immunosorbent assay) according to Example 1 of the present disclosure.

FIG. 4 shows an SDS-PAGE (sodium dodecyl sulfate-polyacrylamide gel electrophoresis) image of the nanobody-Fc fusion protein according to Example 1 of the present disclosure.

FIG. 5 shows the results of gel filtration chromatography characterizing the binding between the nanobody-Fc fusion protein and TD1 according to Example 1 of the present disclosure.

FIG. 6 shows the ELISA results characterizing the binding between the nanobody-Fc fusion protein and CD150 according to Example 1 of the present disclosure.

FIG. 7 shows the affinity between the nanobody and TD1 characterized by BLI (bio-layer interferometry) according to Example 1 of the present disclosure.

FIG. 8 shows the inhibition of mouse tumor proliferation by the anti-CD150 antibody according to Example 2 of the present disclosure.

### DETAILED DESCRIPTION

The embodiments of the present disclosure will be described in detail below with reference to examples as illustrated in the accompanying drawings, throughout which same or similar elements, or elements having same or similar functions, are denoted by same or similar reference numerals.

It should be noted that, terms “first” and “second” are merely used for a descriptive purpose, but cannot be understood as an indication or implication of relative importance or an implied specification of the quantity of indicated technical features. Thus, features defined with the “first” and “second” may explicitly or implicitly include one or more features. Further, in the descriptions of the present disclosure, unless otherwise noted, the meaning of “more” refers to two or more than two.

As used herein, the term “include” or “comprise” is an open inclusion expression, i.e., including the contents specified in the present disclosure, but not excluding other contents.

As used herein, the terms “optionally” and “optional” generally mean that subsequent events or status may occur but not necessarily occur; and the descriptions include conditions under which the events or status occur, and conditions under which the events or status do not occur.

As used herein, the term “antibody” generally refers to an antibody that can recognize one or more antigenic epitopes, including but not limited to monoclonal antibodies, polyclonal antibodies, multimeric antibodies, and CDR-grafted antibodies.

As used herein, the terms “CDR-grafted antibody” and “modified antibody” both refer to antibodies in which the CDRs of a monoclonal antibody from one species are grafted onto the variable region of an antibody from another species. For example, the CDRs of a murine monoclonal antibody may be grafted onto the variable region of a human antibody to replace the CDRs of the human antibody, thus conferring on the human antibody the antigen-binding specificity of the murine monoclonal antibody while reducing immunogenicity. It should be noted that both the polyclonal antibody and the monoclonal antibody in the present disclosure may be CDR-grafted antibodies.

As used herein, the term “monoclonal antibody” refers to an antibody that specifically recognizes a single antigenic epitope. Common monoclonal antibodies include two light chains with lighter molecular weight and two heavy chains with heavier molecular weight. The heavy chain (H chain) is linked to the light chain (L chain) by disulfide bonds to form a tetrapeptide chain molecule. The amino-terminal (N-terminal) region of the heavy and light chains exhibits high sequence variability and is referred to as the variable region (V region), while the carboxy-terminal (C-terminal) region is relatively conserved and is referred to as the constant region (C region). The V regions of the L chain and the H chain are referred to as VL and VH, respectively. Monoclonal antibodies may also exist in the form of small antibody fragments, which mainly include Fab antibodies, Fv antibodies, single-chain antibodies, single-domain antibodies, and minimum recognition units.

As used herein, the term “Fab antibody” generally refers to an antibody fragment containing only the Fab portion, which includes the VH and CH1 of the heavy chain and the complete light chain. The light and heavy chains are connected via a disulfide bond.

As used herein, the term “Fv antibody” refers to an antibody fragment composed solely of the variable regions of the light chain (VL) and the heavy chain (VH), which are associated via non-covalent interactions. The Fv fragment represents the smallest functional unit of an antibody that retains full antigen-binding capability.

As used herein, the term “single-domain antibody”, “nanobody” and “VHH antibody” are used interchangeably and refer to antigen-binding immunoglobulin (variable) domains of first described “heavy-chain antibodies” that naturally lack light chains (Hamers-Casterman C, Atarhouch T, Muyldermans S, Robinson G, Hamers C, Songa E B, Bendahman N, Hamers R.: “Naturally occurring antibodies devoid of light chains”; Nature 363,446-448(1993)). Such antibody includes only a heavy chain variable region (VH) and conventional CH2 and CH3 regions, and specifically binds to an antigen through the heavy chain variable region.

As used herein, the term “single-chain antibody” generally refers to an antibody composed of a heavy chain variable region (VH) and a light chain variable region (VL) that are linked by a linking peptide.

As used herein, the terms “minimum recognition unit” and “MRU” both refer to an antibody consisting solely of a single CDR, which has a extremely small molecular weight accounting for only about 1% of a full-length antibody.

As used herein, the term “polyclonal antibody” refers to an antibody that can recognize multiple antigenic epitopes, e.g., an antibody that can recognize two antigenic epitopes (referred to as a bispecific antibody), an antibody that can recognize three antigenic epitopes, or an antibody that can recognize four antigenic epitopes. It is understood in a broad sense and the specific structure is not limited as long as it can recognize multiple antigenic epitopes.

As used herein, the term “immunogenicity” refers to the ability of a substance to elicit an immune response, i.e., the capability of an antigen to stimulate specific immune cells, leading to their activation, proliferation, and differentiation, and ultimately resulting in the production of immune effector substances such as antibodies and sensitized lymphocytes.

As used herein, the term “fragment” refers to a target protein or polypeptide, as well as a target protein or polypeptide having an N-terminal (N-terminus) or C-terminal (C-terminus) truncation, and/or internal deletions.

As used herein, the terms “identity”, “homology” and “similarity” are all used for describing the percentage of identical amino acids or nucleotides between two amino acid or nucleotide sequences, respectively, relative to a reference sequence. This percentage is determined using conventional methods, such as those described in Ausubel et al., eds. (1995), Current Protocols in Molecular Biology, Chapter 19 (Greene Publishing and Wiley-Interscience, New York); and the ALIGN program (Dayhoff (1978), Atlas of Protein Sequence and Structure 5: Suppl. 3 (National Biomedical Research Foundation, Washington, D.C.)). Various algorithms are applicable to sequence alignment and sequence identity determination, including: the homology alignment algorithm of Needleman et al. (1970), J. Mol. Biol. 48:443; the local homology algorithm of Smith et al. (1981), Adv. Appl. Math. 2:482; the similarity search method of Pearson et al. (1988), Proc. Natl. Acad. Sci. 85:2444; the Smith-Waterman algorithm (Meth. Mol. Biol. 70:173-187 (1997)); and BLASTP, BLASTN, and BLASTX algorithms (see Altschul et al. (1990), J. Mol. Biol. 215:403-410). Computer programs implementing these algorithms are commercially available and include, but are not limited to: ALIGN or MegAlign software (DNASTAR); WU-BLAST-2 (Altschul et al., Meth. Enzym. 266:460-480 (1996)); GAP, BESTFIT, BLAST, FASTA, and TFASTA, all available as part of the Genetics Computer Group (GCG) package, version 8, Madison, Wisconsin, USA; and the CLUSTAL program available in the PC/Gene package from Intelligenetics, Mountain View, California.

As used herein, the term “at least 80% identity” refers to identity to the reference sequence of at least 80%, such as 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.9%.

As used herein, the term “expression vector” generally refers to a nucleic acid molecule that can be inserted into a suitable host for self-replication, and transfers the inserted nucleic acid molecule into host cells and/or between host cells. The expression vector may be primarily used for introducing DNA or RNA into cells, for replicating DNA or RNA, or for expressing DNA or RNA through transcription and/or translation. The expression vector may also possess multiple of the functions as described above. The expression vector may be a polynucleotide that can be transcribed and translated into a polypeptide when introduced into a suitable host cell. Generally, the expression vector can produce a desired expression product by culturing suitable host cells containing the expression vector.

As used herein, the term “recombinant cell” generally refers to a cell that has been genetically modified or recombined using genetic engineering techniques or cell fusion techniques to acquire a stable and inheritable unique trait. The term “host cell” refers to a prokaryotic cell or eukaryotic cell into which a recombinant expression vector can be introduced. As used herein, the term “transformed” or “transfected” refers to the introduction of a nucleic acid (e.g., a vector) into a cell by various techniques known in the art. Suitable host cells can be transformed or transfected with the DNA sequences of the present disclosure and can be used for expression and/or secretion of a target protein. Examples of suitable host cells that can be used in the present disclosure include immortalized hybridoma cells, NS/0 myeloma cells, 293 cells, Chinese hamster ovary (CHO) cells, HeLa cells, Cap cells (human amniotic fluid-derived cells), and CoS cells.

As used herein, the term “pharmaceutical composition” generally refers to a unit dosage form and can be prepared by any of the methods well known in the pharmaceutical field. All methods include the step of combining an active ingredient with a carrier which constitutes one or more accessory ingredients. In general, the composition is prepared by uniformly and intimately combining the active compound with a liquid carriers, finely divided solid carrier, or both.

As used herein, the term “antigen-binding fragment” refers to an antibody fragment that retains the ability to specifically bind to an antigen.

The embodiments of the present disclosure are explained below with reference to the examples. Those skilled in the art will appreciate that the following examples are merely used for illustrating the present disclosure and should not be construed as limiting the scope of the present disclosure. Where specific techniques or conditions are not specified in the examples, they are conducted according to technologies or conditions described in the document in the art or according to the product manual. The reagents or instruments used without indicating manufacturers are all conventional products purchasable in the market.

### Example 1: Preparation of Anti-CD150 Antibody

### 1. Construction of Expression Vector

The coding sequence of the extracellular domain of CD150 was amplified by PCR from the full-length CD150 gene synthesized by a commercial provider. The amplified sequence fragment was inserted into the PTT5-hFc recombinant plasmid via restriction enzyme digestion at the SalI and XbaI sites. The ligation product was transformed into E. coli DH5α, plated, and incubated overnight. The resulting colonies were screened by PCR. The PCR-positive clones were subjected to DNA sequencing. The sequencing results indicated that the vector ptt5-CD150-hFc was successfully constructed.

### 2. Expression, Purification, and Characterization of Immunogen

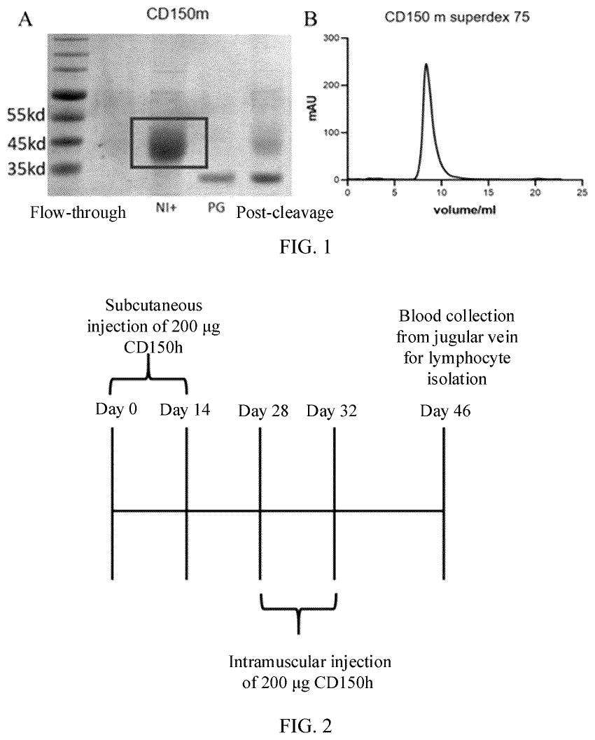

First, the ptt5-CD150-hFc plasmid was transfected into 293F cells using PEI at a 2:1 ratio. The cells were cultured at 37° C., 5% CO₂, and 120 rpm for 4 days. The culture supernatant was collected by centrifugation at 4000 g for 20 minutes. The CD150 protein with an hFc fragment was purified using a Protein A affinity column, eluted with 0.1 M acetic acid, and neutralized using Tris-HCL at pH=8.0, to obtain CD150 protein with human Fc. Next, the hfc fragment was cleaved overnight at 4° C. using TEV protease. The resulting protein was concentrated with a 10 KD ultrafiltration tube, and washed with PBS. The purified CD150 protein was verified by SDS-PAGE. Finally, the protein was filtered through a Superdex 75 molecular sieve to determine its size and state. The CD150 protein was obtained. As shown in FIG. 1, the results indicated that the purified CD150h protein was obtained and confirmed to be a monomer.

3. Immunization of Alpacas with CD150 Protein

Alpacas were immunized with the recombinantly expressed CD150 protein, and the immunization process is shown in FIG. 2. In the examples of the present disclosure, four rounds of immunizations were performed in total, in which the first two via subcutaneous injection and the last two via intramuscular injection. The interval between the two subcutaneous injections was 14 days, and the interval between the two intramuscular injections was 14 days. Peripheral blood was collected from the jugular veins of the alpacas 14 days after the last intramuscular injection. In the examples of the present disclosure, the dosage of the CD150 protein for each immunization of the alpacas was 200 μg.

### 4. Screening of Nanobody

a. The collected alpaca blood was slowly layered over an equal volume of Ficol solution and centrifuged at 400 g, 25° C. for 20 minutes with a deceleration setting adjusted from 6 to 2. The middle lymphocyte layer was harvested, washed with DMEM medium, and centrifugated to collect the cell pellet. Total RNA was extracted using an RNA extraction kit and reverse-transcribed into cDNA.

b. Using the cDNA as a template, the VHH sequence was amplified with specific primers.

Forward primer F1:
(SEQ ID NO: 7)
5′-GCTGCACAGCCTGCTATGGCACAGKTGCAGCTCGTGGAGTCTGGGG
G-3′;
Reverse primer R1:
(SEQ ID NO: 8)
5′-GAGTTTTTGTTCGGCTGCTGCTGAGGAGACGGTGACCTGGGTCCC
C-3′.

Using 0.5 μL of cDNA as a template, the VHH (alpaca single-domain antibody) fragment was amplified with two primers. The DNA polymerase used was PrimerStar 2× MAX polymerase. The reaction system was 50 μL. The annealing temperature was 55° C. PCR products were confirmed via gel electrophoresis and recovered using a gel extraction kit.

Using pR2 phagemid as a template, pR2 phagemid was amplified with specific primers.

Forward primer F2:
(SEQ ID NO: 9)
5′-AGCAGCCGAACAAAAACTCATCTCAGAAGAG-3′;
Reverse primer R2:
(SEQ ID NO: 10)
5′-CCATAGCAGGCTGTGCAGCATAGAAAGGTACCACTAAAGGAATTG
C-3′.

Using 10 ng of pR2 plasmid as a template, PCR amplification was performed using the above primers. The DNA polymerase used was PrimerStar 2×MAX. The annealing temperature was 53° C. A total of two 50 μL systems were required for the amplification. The products were recovered using a PCR purification kit and eluted in 50 μL of ddH2O.

pR2 phagemid and the VHH fragment were ligated using seamless cloning at a molar ratio of 1:4. The ligation products were electroporated into TG1 competent cells and cultured at 37° C., 200 rpm for 1 hour. 0.2 μL and 0.02 μL (by dilution) of the bacterial solution were respectively spread onto 10 cm solid plates. After culturing for 13 hours, the number of colonies was counted and to calculate the size of the constructed antibody library. The remaining bacterial liquid was centrifugated and spread onto a 150 mm solid plate and cultured at 37° C. for 13 hours. Then, the colonies were scraped off, snap-frozen in liquid nitrogen and stored at −80° C. This served as the nanobody library.

c. The cryopreserved antibody library bacteria were revived, and KM13 helper phage was added to assist growth and proliferation of M13 phage. The bacterial culture supernatant was collected to measure phage titer, which served as the amplified phage. CD150 antigen was coated on immunoassay plates at a concentration of 0.1 mg/mL, and 1×10¹¹ pfu of the above-amplified phage were added, followed by incubation at room temperature for 1 hour. Phages specifically bound to the CD150 antigen were eluted with trypsin and used to infect TG1 bacteria. 50 μL and 5 μL of the infected bacterial solution were spread onto solid plates, and the total number of colonies was recorded.

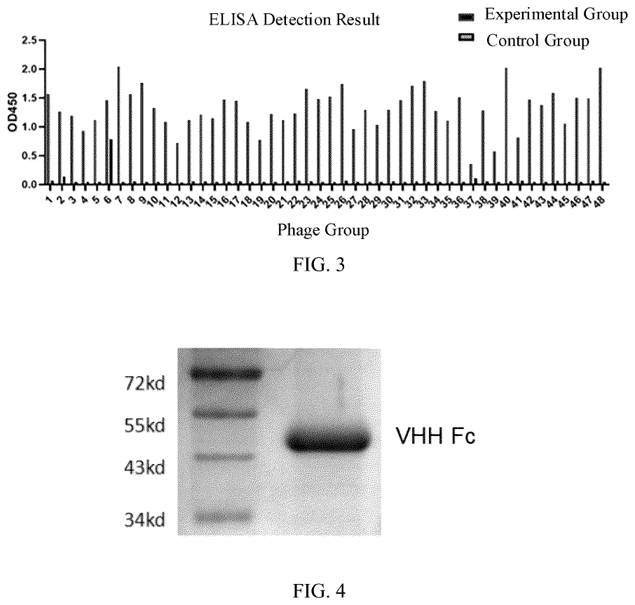

d. 95 single colonies were randomly picked from the above plates and activated overnight. KM13 helper phage was added, and the supernatant after lysis was collected by centrifugation, which served as monoclonal phages. CD150 antigen was coated onto a 96-well immunoassay plates at a concentration of 1 μg/mL, and the monoclonal phage solution prepared above was added to each well, followed by incubation at room temperature for 1 hour. Phages bound to the CD150 antigen were captured using HRP-conjugated anti-M13 antibody, and TMB substrate was added for color development for 5 minutes. The reaction was terminated with 1 M H₂SO₄ solution. A microplate reader was used to record OD450 values. The result is shown in FIG. 3, indicating that ELISA results confirmed that the control group was not coated with CD150, while the experimental group was coated with the CD150 protein.

e. Clones with the OD450 values greater than 1 were selected for sequencing analysis.

f. After analyzing the antibody sequences and excluding duplicate clones, one nanobody sequence was obtained.

### 5. Expression and Purification of CD150 Nanoantibody and Fc Fusion Protein

The nanobody sequence of the present disclosure was constructed into a mammalian expression vector pTT5 with a signal peptide, and fused with human IgG1 Fc for expression. The Fc fragment was expressed at the C-terminus and can be cleaved with TEV protease to obtain the nanobody. The recombinant plasmid was transfected into HEK 293 F cells, and the culture supernatant was collected. The nanobody Fc fusion protein was purified using a Protein A affinity chromatography column. As shown in FIG. 4, a high-purity nanobody Fc fusion protein was finally obtained.

The nucleotide sequence of the CD150 antigen described in the examples of the present disclosure is as set forth in SEQ ID NO: 6:

(SEQ ID NO: 6)
ATGGCAAGCTACGGAACAGGTGGGCGCATGATGAACTGCCCAAAGATT
CTCCGGCAGTTGGGAAGCAAAGTGCTGCTGCCCCTGACATATGAAAGG
ATAAATAAGAGCATGAACAAAAGCATCCACATTGTCGTCACAATGGCA
AAATCACTGGAGAACAGTGTCGAGAACAAAATAGTGTCTCTTGATCCA
TCCGAAGCAGGCCCTCCACGTTATCTAGGAGATCGCTACAAGTTTTAT
CTGGAGAATCTCACCCTGGGGATACGGGAAAGCAGGAAGGAGGATGAG
GGATGGTACCTTATGACCCTGGAGAAAAATGTTTCAGTTCAGCGCTTT
TGCCTGCAGTTGAGGCTTTATGAGCAGGTCTCCACTCCAGAAATTAAA
GTTTTAAACAAGACCCAGGAGAACGGGACCTGCACCTTGATACTGGGC
TGCACAGTGGAGAAGGGGGACCATGTGGCTTACAGCTGGAGTGAAAAG
GCGGGCACCCACCCACTGAACCCAGCCAACAGCTCCCACCTCCTGTCC
CTCACCCTCGGCCCCCAGCATGCTGACAATATCTACATCTGCACCGTG
AGCAACCCTATCAGCAACAATTCCCAGACCTTCAGCCCGTGGCCCGGA
TGCAGGACAGACCCCTCAGAAACAAAACCATGGGCAGTGTATGCTGGG
CTGTTAGGGGGTGTCATCATGATTCTCATCATGGTGGTAATACTACAG
TTGAGAAGAAGAGCCACCTTGACTACTACCAACCAATACTGGAGTCAA
AATGTCCTGACCCAAGACCAGGAGAGATGCCCCGGCTGCCTTCCCATG
GTAAAACGAACCATTACCAGACAACAGTGGAAAAAAAAAGCCTTACGA
TCTATGCCCAAGTCCAGAAACCAGGTCCTCTTCAGAAGAAACTTGACT
CCTTCCCAGCTCAGGACCCTTGCACCACCATATATGTTGCTGCCACAG
AGCCTGTCCCAGAGTCTGTCCAGGAAACAAATTCCATCACAGTCTATG
CTAGTG.

### 6. Characterization of CD150 Nanobody

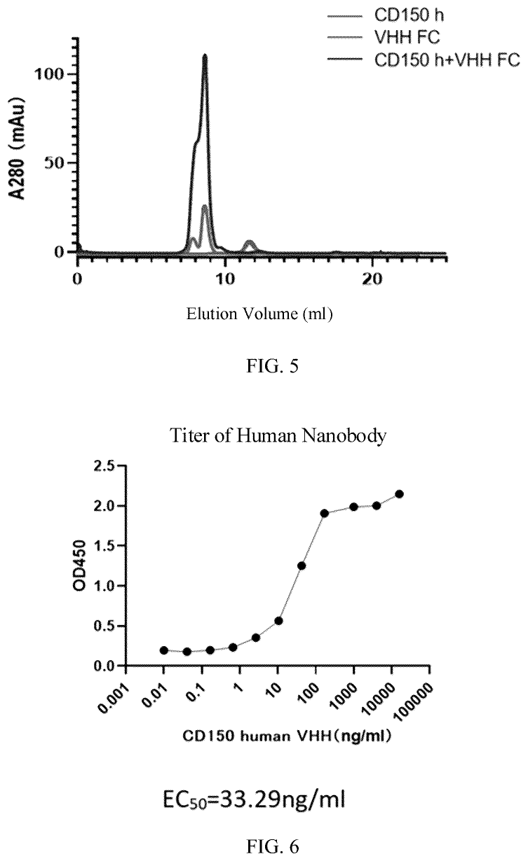

a. Binding between the nanobody Fc fusion protein and CD150 was characterized by gel filtration chromatography. The nanobody Fc fusion protein and CD150 protein were mixed at a molar ratio of 2:1, incubated for 1 hour, and then loaded onto a Superdex 75 gel filtration chromatography column. The absorbance at 280 nm was recorded, and a chromatogram was plotted. In addition, a nanobody Fc fusion protein control and a CD150 protein control were set up. As shown in FIG. 5, the results confirmed binding between the CD150 protein and the antibody.

b. Binding between the nanobody Fc fusion protein and SARS-CoV-2 RBD was characterized by non-competitive ELISA. CD150 protein at a concentration of 10 μg/mL was coated onto immunoassay plates, and the nanobody Fc fusion protein with a gradient dilution of 1:4 was added in sequence and incubated at room temperature for 1 hour. HRP-conjugated anti-IgG1 Fc antibody was added, followed by a substrate solution and a termination solution to measure absorbance of OD450. As shown in FIG. 6, the obtained antibody showed an EC₅₀ of 33.29 ng/ml, indicating that the obtained antibody had a high titer.

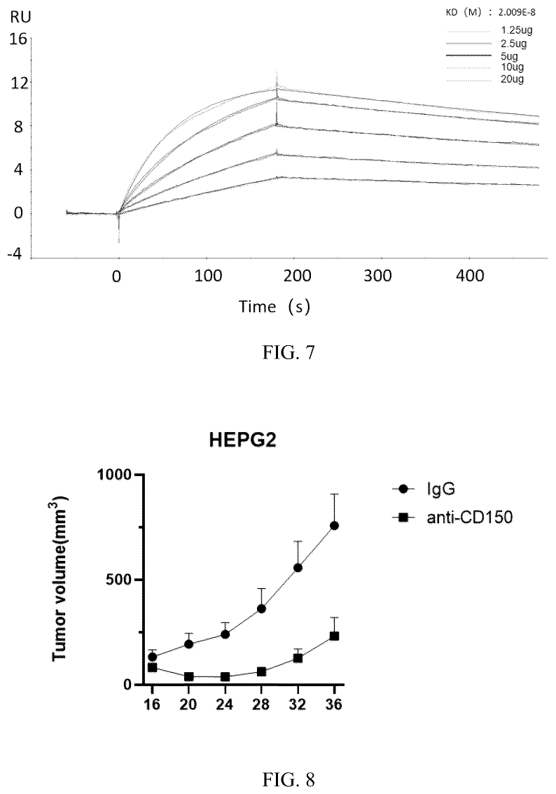

c. BLI was used to characterize the affinity of the nanobody to CD150. In order to characterize the affinity of the nanobody to CD150, CD150 was first labeled with biotin to obtain biotinylated CD150 protein (biotin-CD150). Then, the biotin-CD150 was immobilized on a streptavidin (SA) biosensor, and different concentration gradients of the nanobody Fc fusion protein were tested to detect affinity of the biotin CD150 and the nanobody Fc fusion protein. As shown in FIG. 7, the results indicated that the antibody showed strong affinity for the antigen.

### Example 2: Inhibition of Hepatocellular Carcinoma Proliferation in Mice by Anti-CD150 Antibody

According to the examples of the present disclosure, the anti-CD150 antibody of the present disclosure was evaluated using a mouse model with hepatocellular carcinoma. The specific steps were as follows:

Humanized mice were treated using the CD150 blocking antibody of the present disclosure was used. In the control group, the same dose of IgG was administered as a control. Twelve NSG mice (an immunodeficient mouse model) were used. The mice were anesthetized with 2.5% isoflurane and fixed using a mouse restrainer. The tails of the mice were straightened, and thus their tail veins on both sides were clearly visible. 1×10⁷ PBMC cells were infused into the mice via their tail veins, with a total volume of approximately 100 μl per infusion. On day 6, the mice were randomly divided into two groups, 6 vs. 6. HEPG2 cells (hepatocellular carcinoma cells) were subcutaneously inoculated into the mice, with 5×10⁶ cells per mouse in a total volume of 100 μl. Intraperitoneal administration was initiated from day 13. The treatment group was administered 10 mg/kg of the antibody, while the control group received an equal volume of IgG, with a total volume of 100 μl. A total of 5 treatments were given at 3-day intervals. A tumor volume was measured starting from day 16, with measurements taken every two days. When the tumor volume of mice exceeded 1000 mm, they were considered deceased, and subsequent data were no longer recorded. After the administration, a significant reduction in a tumor growth rate was observed (FIG. 8), indicating that the anti-CD150 antibody can block CD150, thereby achieving the purpose of tumor treatment.

In the present description, descriptions of the reference terms such as “one embodiment”, “some embodiments”, “examples”, “specific examples” and “some examples” mean that, specific features, structures, materials or characteristics described in combination with the embodiments or examples are included in at least one embodiment or example of the present disclosure. In the present description, schematic expressions of the above terms are unnecessarily specified at the same embodiment or example. Moreover, the specific features, structures, materials or characteristics described herein may be in combination in any of or more embodiments or examples in an appropriate mode. In addition, in absence of mutual contradiction, different embodiments or examples described in the present description and features of the different embodiments or examples may be in integration and combination by those skilled in the art.

What is claimed is:

1. An antibody or antigen-binding fragment, comprising a heavy chain variable region CDR sequence selected from at least one of SEQ ID NO: 1 to SEQ ID NO: 3 or amino acid sequences having at least 80% identity to SEQ ID NO: 1 to SEQ ID NO: 3.

2. The antibody or antigen-binding fragment according to claim 1, comprising:
a heavy chain variable region CDR1 sequence, a heavy chain variable region CDR2 sequence, and a heavy chain variable region CDR3 sequence as set forth in amino acid sequences of SEQ ID NO: 1, SEQ ID NO: 2, and SEQ ID NO: 3, respectively, or amino acid sequences having at least 80% identity to SEQ ID NO: 1, SEQ ID NO: 2, and SEQ ID NO: 3, respectively.

3. The antibody or antigen-binding fragment according to claim 1, further comprising a heavy chain framework region sequence, wherein at least a portion of the heavy chain framework region sequence is from at least one of a murine antibody, a human antibody, a primate antibody, an alpaca antibody, or a mutant thereof.

4. The antibody or antigen-binding fragment according to claim 2, comprising a heavy chain variable region having an amino acid sequence as set forth in SEQ ID NO: 4.

5. The antibody or antigen-binding fragment according to claim 1, further comprising a heavy chain constant region, wherein at least a portion of the heavy chain constant region is from at least one of a murine antibody, a human antibody, a primate antibody, or a mutant thereof.

6. The antibody or antigen-binding fragment according to claim 5, wherein the heavy chain constant region is from an alpaca antibody.

7. The antibody or antigen-binding fragment according to claim 1, wherein the antibody or antigen-binding fragment comprises a monoclonal antibody or a polyclonal antibody.

8. The antibody or antigen-binding fragment according to claim 7, wherein the monoclonal antibody comprises at least one of a Fab antibody, an Fv antibody, a single-chain antibody, a single-domain antibody, and a minimum recognition unit.

9. The antibody or antigen-binding fragment according to claim 8, wherein the antibody has an amino acid sequence as set forth in SEQ ID NO: 5.

10. A recombinant protein, comprising the antibody or antigen-binding fragment according to claim 1.

11. The recombinant protein according to claim 10, further comprising at least one selected from a bioactive protein or fragment thereof, and a bioactive polypeptide or fragment thereof.

12. The recombinant protein according to claim 11, wherein the bioactive protein or fragment thereof comprises at least one selected from a protein tag, a protein toxin or fragment thereof, a tumor necrosis factor or fragment thereof, an interferon or fragment thereof, a biological response modifier or fragment thereof, and an Fc fragment.

13. A nucleic acid molecule, encoding the antibody or antigen-binding fragment according to claim 1.

14. An expression vector, carrying the nucleic acid molecule according to claim 13.

15. A recombinant cell, expressing the antibody or antigen-binding fragment according to claim 1.

16. A pharmaceutical composition, comprising the antibody or antigen-binding fragment according to claim 1.

17. The pharmaceutical composition according to claim 16, further comprising a pharmaceutically acceptable carrier or excipient.

18. A kit, comprising the antibody or antigen-binding fragment according to claim 1.

19. A method for treating or preventing a disease, the method comprising:
administering to a subject the antibody or antigen-binding fragment according to claim 1.

20. The method according to claim 19, wherein the disease comprises lung cancer, gastric cancer, colorectal cancer, liver cancer, breast cancer, esophageal cancer, or thyroid cancer.


SEQUENCE LISTING (10 sequences)

SEQ ID NO: 1 - protein, 10 aa, synthetic construct
GRAFSDYAVG

SEQ ID NO: 2 - protein, 14 aa, synthetic construct
EFVATISWSGFSTY

SEQ ID NO: 3 - protein, 18 aa, synthetic construct
ADRNGTSFPYIRRDEYDY

SEQ ID NO: 4 - protein, 123 aa, synthetic construct
QVQLVESGGGLVQAGGSLRLSCAASGRAFSDYAVGWFRQAPGREREFVATISWSGFSTYYADSVKGRFTISRDNAEKTVTLQMNSLKPEDTAVYYCAADRNGTSFPYIRRDEYDYWGQGTQVT

SEQ ID NO: 5 - protein, 123 aa, synthetic construct; identical to SEQ ID NO: 4

SEQ ID NO: 6 - DNA, 1014 bp, synthetic construct
  source 1..1014: mol_type = other DNA
atggcaagctacggaacaggtgggcgcatgatgaactgcccaaagattctccggcagttgggaagcaaagtgctgctgcccctgacatatgaaaggataaataagagcatgaacaaaagcatccacattgtcgtcacaatggcaaaatcactggagaacagtgtcgagaacaaaatagtgtctcttgatccatccgaagcaggccctccacgttatctaggagatcgctacaagttttatctggagaatctcaccctggggatacgggaaagcaggaaggaggatgagggatggtaccttatgaccctggagaaaaatgtttcagttcagcgcttttgcctgcagttgaggctttatgagcaggtctccactccagaaattaaagttttaaacaagacccaggagaacgggacctgcaccttgatactgggctgcacagtggagaagggggaccatgtggcttacagctggagtgaaaaggcgggcacccacccactgaacccagccaacagctcccacctcctgtccctcaccctcggcccccagcatgctgacaatatctacatctgcaccgtgagcaaccctatcagcaacaattcccagaccttcagcccgtggcccggatgcaggacagacccctcagaaacaaaaccatgggcagtgtatgctgggctgttagggggtgtcatcatgattctcatcatggtggtaatactacagttgagaagaagagccaccttgactactaccaaccaatactggagtcaaaatgtcctgacccaagaccaggagagatgccccggctgccttcccatggtaaaacgaaccattaccagacaacagtggaaaaaaaaagccttacgatctatgcccaagtccagaaaccaggtcctcttcagaagaaacttgactccttcccagctcaggacccttgcaccaccatatatgttgctgccacagagcctgtcccagagtctgtccaggaaacaaattccatcacagtctatgctagtg

SEQ ID NO: 7 - DNA, 47 bp, synthetic construct
  source 1..47: mol_type = other DNA
gctgcacagcctgctatggcacagktgcagctcgtggagtctggggg

SEQ ID NO: 8 - DNA, 46 bp, synthetic construct
  source 1..46: mol_type = other DNA
gagtttttgttcggctgctgctgaggagacggtgacctgggtcccc

SEQ ID NO: 9 - DNA, 31 bp, synthetic construct
  source 1..31: mol_type = other DNA
agcagccgaacaaaaactcatctcagaagag

SEQ ID NO: 10 - DNA, 46 bp, synthetic construct
  source 1..46: mol_type = other DNA
ccatagcaggctgtgcagcatagaaaggtaccactaaaggaattgc